(19) United States
(12) Patent Application Publication
(10) Pub. No.: US 2014/0248272 A1
(43) Pub. Date: Sep. 4, 2014

(54) RECOMBINANT PROTEINS FOR USE IN VACCINE, ANTIBODIES AGAINST SAID PROTEINS, AND DIAGNOSTIC AND THERAPEUTIC METHODS INCLUDING THE SAME

(71) Applicant: Vicia Animal Health Science AB, Sollentuna (SE)

(72) Inventors: Anna ROSANDER, Uppsala (SE); Märit PRINGLE, Knivsta (SE)

(21) Appl. No.: 14/196,228

(22) Filed: Mar. 4, 2014

Related U.S. Application Data
(63) Continuation-in-part of application No. 13/576,032, filed on Jul. 30, 2012, now Pat. No. 8,703,432.
(60) Provisional application No. 61/298,960, filed on Jan. 28, 2010.
(60) Provisional application No. 61/360,986, filed on Jul. 2, 2010.

(51) Int. Cl. C07K 14/20; C07K 16/12; G01N 33/569

(52) U.S. Cl. CPC C07K 14/20; G01N 33/56911; C07K 16/1207
USPC 424/139.1; 530/350; 530/324; 530/326; 424/190.1; 436/501; 435/7.95; 435/7.92; 530/387.9; 435/7.32; 536/23.7; 435/320.1; 435/69.3; 435/252.33

(57) ABSTRACT

The present invention relates to proteins and/or fragments and derivatives thereof and their use as vaccines and in biotechnological methods. The vaccines particularly include immunogenic proteins in Treponema spp. isolated from digital dermatitis in cattle. The present invention further relates to antibodies raised against said proteins or fragments thereof, and the use of said proteins in diagnostic methods in which antibodies are detected as a sign of digital dermatitis in cattle.

### TECHNICAL FIELD

The present invention relates to proteins and/or fragments and derivatives thereof and their use as vaccines and in biotechnological methods. The vaccines particularly include immunogenic proteins in Treponema spp. isolated from digital dermatitis in cattle. The present invention further relates to antibodies raised against said proteins or fragments thereof, and the use of said proteins in diagnostic methods in which antibodies are detected as a sign of digital dermatitis in cattle.

### BACKGROUND

Digital dermatitis (DD) is a contagious claw disease causing lameness in cattle, most commonly seen in intensive dairy production. The disease was first described in 1974 in Italy. In Sweden the first herd with DD was described recently (Hillstrom and Bergsten, 2005) whereas previously only sporadic, atypical cases have been reported (Manske et al., 2002). There is a strong connection between wet/dirty claw environments and the occurrence of DD (Rodriguez-Lainz et al., 1996), for example in cubicle systems where accumulation of faeces and urine on the alleys is a typical hygienic problem. Besides being an animal welfare problem, economic losses due to reduced milk production and weight loss are associated with DD (Losinger, 2006).

The rapid response to antibiotic treatment of DD lesions strongly supports a bacterial cause. Many bacteria of different genera, such as Treponema, Fusobacterium, Dichelobacter, Prevotella, and Porphyromonas have been isolated from DD lesions and a polymicrobial cause is often discussed. However, there is strong circumstantial evidence that Treponema spp. are central in the aetiology of DD. As early as 1964 spirochetes were observed in smears from different variants of “foot-rot” manifestations in cattle (Gupta et al., 1964). Another early observation of spirochetes was made 1988 when DD was described for the first time in the UK (Blowey and Sharp, 1988). The first spirochete cultures from DD were reported 1995 (Walker et al., 1995). In histological preparations from DD lesions treponemes are found invading the deeper layers of epidermis (Moter et al., 1998). Additionally a humoral immune response against Treponema spp. has been demonstrated in infected cattle (Walker et al., 1997; Trott et al., 2003). Successful experimental transmission of the disease through inoculation with fresh scrapings from DD lesions was described in 1996 (Read and Walker, 1996). It was also confirmed by histopathology that spirochetes invaded the tissue 1-2 weeks after inoculation (Read et al., 1998).

Several phylotypes of Treponema can be present in the same lesion. Different phylotypes have been isolated from the same animal (Walker et al., 1995; Evans et al., 2008) and by cloning and sequencing of 16S rRNA genes, five different phylotypes were identified in a pooled sample from four cows (Choi et al., 1997). It has also been demonstrated by fluorescence in situ hybridization on biopsies from DD lesions that the distribution in the dermal layers differs between phylotypes (Moter et al., 1998). The Treponema phagedenis-like phylotype was located mainly in the stratum corneum and stratum spinosum. Some phylotypes have not yet been reported as cultured. Recently the Treponema phagedenis-like phylotype has been indicated in several studies to be a key agent in the pathogenesis of DD (Klitgaard et al. 2008, Nordhoff et al. 2008, Yano et al. 2009).

In countries where DD is widespread, footbaths containing antibiotics are often used. These footbaths rapidly become contaminated with faeces and dirt and hence function as large selective cultures of antibiotic resistant bacteria. In Sweden tetracyclines are used, but only for topical treatment of individual animals since on herd level footbaths with copper sulphate are recommended.

To date no commercial vaccine or serologic test for DD is available. A humoral response against Treponema spp. has been shown in cattle with DD and used for whole cell lysate ELISA investigations in research (Demirkan et al. 1999, Trott et al. 2003, Vink et al. 2009, Walker et al. 1997). Novartis produced a whole cell lysate DD vaccine (TrepShield) for the USA market for some years in the early 2000s (Berry et al. 2004, Keil et al. 2002).

Technologies and strategies for development of vaccines are described in i.a. Vaccine Design: Innovative Approaches and Novel Strategies (Caister Academic Press, 2011) and Vaccines: From Concept to Clinic: A Guide to the Development and Clinical Testing of Vaccines for Human Use (Informa Healthcare, 1998). The use of a recombinant protein as a vaccine is described in Erdile et al. 1997.

### SUMMARY OF THE INVENTION

The present invention aims at providing efficient methods for diagnosis of and immuno-protection against dermatitis in animals, particularly digital dermatitis in ruminants, as well as products for said purposes.

The present invention revolves around immunogenic proteins in Treponema spp. isolated from digital dermatitis in cattle, and more specifically to recombinant proteins.

In a first aspect, the present invention relates to isolated Treponema phagedenis-like proteins, TmpA, Ttm, PrrA, VpsA and VpsB with amino acid sequence according to SEQ ID NO:2, SEQ ID NO:4, SEQ ID NO:6, SEQ ID NO:18 and SEQ ID NO:20, respectively, to fragments and derivatives thereof capable of inducing an immune response to Treponema spp., and to fragments and derivatives capable of binding to antibodies produced by a subject in an immune response against said protein, as further defined below.

In one embodiment of the invention, the Treponema proteins, and fragments and derivatives thereof, are recombinantly produced.

In one aspect, the invention relates to nucleic acid molecules encoding the proteins, fragments and derivatives according to the invention.

The invention also relates to the use of said proteins, fragments and derivatives thereof in veterinary medicine, specifically as a vaccine for prevention of digital dermatitis.

In a further aspect, the present invention provides a veterinary vaccine for protection against digital dermatitis comprising one or more of said recombinant proteins and/or active fragments thereof, and conventional and suitable adjuvants. Such a vaccine may or may not further include other Treponema immunogens or whole cell lysates of different Treponema spp. in a vaccine for a broader immune response.

According to another aspect, the present invention also relates to a method for prevention of digital dermatitis in animals comprising the step of administering said vaccine to an animal in need thereof.

According to a still further aspect, there is provided a method of detecting presence of antibodies against Treponema spp. in a sample in which said recombinant proteins and/or active fragments are used to detect the presence of antibodies against Treponema spp. in said sample.

An additional aspect of the invention is the diagnostic use of one or more isolated Treponema phageden is-like proteins, TmpA, Ttm, PrrA, VpsA and VpsB which in an advantageous embodiment were identified using a typical promoter sequence, which is described in detail in the Experimental part below. In one embodiment, a panel of proteins is used such as at least two, three, four or five of the above-mentioned proteins. As the skilled person will appreciate, such a panel could also include additional Treponema spp. proteins, not defined by sequence in this application.

Thus, according to one aspect, there is provided a method for diagnosis of digital dermatitis in an animal in which said recombinant proteins and/or active fragments are used to detect the presence of antibodies against Treponema spp. in an animal.

In one embodiment of said detection method or diagnostic method, said recombinant proteins and/or active fragments are used in an ELISA (Enzyme-Linked ImmunoSorbant Assay) method.

In one aspect, the present invention relates to antibodies raised against said immunogenic proteins, or immunogenic derivatives or fragments thereof. Such antibodies are useful in treatment of disease caused by Treponema spp. by way of passive immunization and also in various laboratory methods such as immunomagnetic separation of Treponema bacteria.

### BRIEF DESCRIPTION OF THE DRAWINGS

FIG. 1a) and b) is an enzyme-linked immunosorbant assay with recombinant Treponema phagedenis-like strain V1 immunogenic proteins TmpA, Ttm, and PrrA as antigens. The assays were performed with sera from eight dairy cows with acute digital dermatitis (black bars), two cows with no known history of digital dermatitis and five calves 6-7 months of age (gray bars). Horse-radish peroxidase (HRP) conjugated rabbit anti-bovine IgG antibodies (Sigma) (a) or monoclonal 22:26 anti-bovine IgG-HRP antibodies (Svanova Biotech AB) (b) were used as secondary antibodies. Corrected optical density (COD) was measured at 450 nm.

FIG. 2a), b) and c) are sequence alignments of a large number of fragments of proteins according to the invention as described in detail by the three panning experiments using phage display in the Experimental part below. More specifically, FIG. 2a is an alignment of TmpA fragments; FIG. 2b is an alignment of Ttm fragments; and FIG. 2c is an alignment of PrrA fragments.

### DEFINITIONS

An “immunogenic agent”, or “immunogen”, is capable of inducing an immunological response against itself on administration to a patient, optionally in conjunction with an adjuvant.

An “active fragment” or “active derivative” as used in the present specification is a fragment or derivative of a native immunogenic agent, capable of inducing an immunological response against said native immunogenic agent on administration to a patient, optionally in conjunction with an adjuvant. An active fragment or derivative comprises or mimics at least one “epitope” or “antigenic determinant”.

A “binding fragment” or “binding derivative” as used in the present specification is a fragment or derivative of a native immunogenic agent, capable of immunospecific binding to antibodies produced by a subject in an immune response against said native immunogenic agent. A binding fragment or derivative comprises or mimics at least one “epitope” or “antigenic determinant”.

A “derivative” of a protein may be a protein showing substantial sequence homology to the original protein. The sequence homology may be 50% identity or more, such as 65%, 80%, 85%, 90%, 95% or 99% identity in amino acid sequence. The substituted amino acids are preferably conservative substitutions. The substituted amino acids may be natural or non-natural amino acids.

The term “epitope” or “antigenic determinant” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed. (1996).

Antibodies that recognize the same epitope can be identified in a simple immunoassay showing the ability of one antibody to block the binding of another antibody to a target immunogen, or fragment or derivative thereof.

The term “antibody” refers to an intact antibody, or a binding fragment thereof. An antibody may comprise a complete antibody molecule (including polyclonal, monoclonal or chimeric), or comprise an antigen binding fragment thereof. Antibody fragments include F(ab′).sub.2, Fab, Fab′, Fv, Fc, and Fd fragments, and can be incorporated into single domain antibodies, single-chain antibodies, maxibodies, minibodies, intrabodies, diabodies, triabodies, tetrabodies, v-NAR and bis-scFv (See e.g., Hollinger and Hudson, 2005, Nature Biotechnology, 23, 9, 1126-1136).

Sequence listing

| SEQ ID NO | Type | Description |
|---|---|---|
| 1 | DNA | TmpA homolog, |
| | | complete coding sequence |
| 2 | Protein | TmpA homolog, protein |
| 3 | DNA | putative tail tape measure protein, |
| | | complete coding sequence |
| 4 | Protein | putative tail tape measure protein, protein |
| 5 | DNA | putative proline-rich lipoprotein (PrrA), |
| | | complete coding sequence |
| 6 | Protein | putative proline-rich lipoprotein (PrrA), protein |
| 7 | DNA | GSTtmpAF1 (fwd) |
| 8 | DNA | GSTtmpAR1 (rev) |
| 9 | DNA | GSTkallaF1 (fwd) |
| 10 | DNA | GSTkallaR1 (rev) |
| 11 | DNA | GSTPGKEEF1 (fwd) |
| 12 | DNA | GSTPGKEER1 (rev) |
| 13 | DNA | ImpactPGKEEF1 (fwd) |
| 14 | DNA | ImpactPGKEER1 (rev) |
| 15 | DNA | ImpactkallaF1 (fwd) |
| 16 | DNA | ImpactkallaR1 (rev) |
| 17 | DNA | primerSAsekv |

### DETAILED DESCRIPTION

### Further Outline of Aspects and Embodiments of the Invention

The present invention relates in broad terms to immunogenic proteins and protein fragments as well as to a method of identifying novel immunogenic proteins using a suitable promoter region.

In one aspect the present invention relates to an isolated protein having the amino acid sequence according to SEQ ID NO:2 (TmpA), SEQ ID NO:4 (Ttm), SEQ ID NO:6 (PrrA), SEQ ID NO:18 (VpsA) or SEQ ID NO:20 (VpsB), or fragments or derivatives thereof capable of inducing an immune response to said protein, or a fragment or derivative capable of binding to antibodies produced by a subject in an immune response against said protein. Said protein, fragment or derivate may be used in veterinary medicine, such as in prevention of a disease caused by Treponema spp., such as digital dermatitis.

In one embodiment, the invention relates to a protein fragment, which comprises at least one sequence selected from the group consisting of amino acids 77-120 of SEQ ID NO:2; amino acids 922-948 or 689-909 of SEQ ID NO: 4; and amino acids 66-83 or 163-183 of SEQ ID NO:6.

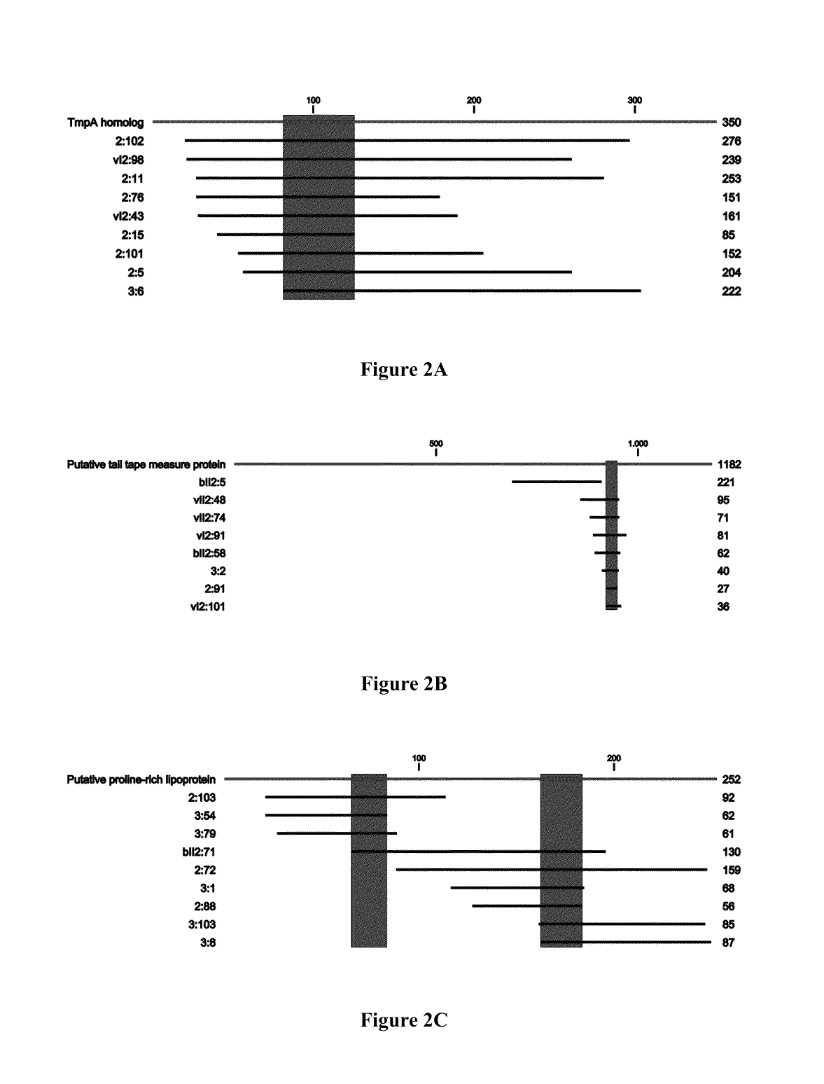

Thus, the present invention shows that each one of the antigenic proteins according to the invention contains certain repeated sequences, known as epitopes, which as shown below (see e.g. FIGS. 2a, 2b and 2c) are capable of binding to antibodies against Treponema. The identification of these fragments enables the skilled person to produce more powerful diagnostic tools using recombinant DNA technology.

As the skilled person will appreciate, for diagnostic or pharmaceutical use, the protein fragments need not be limited to the actual epitopes defined above, but may comprise additional sequence of the original proteins too, as exemplified e.g. by the full length fragments tested as described in the Experimental part.

In an illustrative embodiment, the protein fragment according to the invention is a fragment of SEQ ID NO: 4, as exemplified in the experimental part below. In a specific embodiment, the invention relates to a polypeptide comprising amino acids no 689-970 of SEQ ID NO:4.

The fragments according to the invention may or may not include other amino acids in addition to those specified above, as long as such additional amino acids do not substantially alter the immunogenicity according to the invention.

The present invention also relates to a method of identifying fragments of one of the proteins according to the invention, which fragments are capable of specific binding to antibodies produced by a subject in an immune response against said protein. Once aware of the proteins, the skilled person is able to perform such a method using well known routine technologies.

Screening of antigenicity of Treponema protein fragments according to the invention could for example be performed as follows. Treponemal proteins are first fragmented, and the antigenicity of these fragments investigated in an array. Suitable array formats are well known to the skilled person. For example, protein specific phage display libraries are generated, e.g. as described in the experimental part below but using one specific gene for one library instead of whole genomic DNA. Such phage libraries can be selected against antibodies to identify fragments, which constitute specific epitopes. Specific PCR products, based on results from using the protein specific phage libraries, or PCR products corresponding to random fragments may be produced. The PCR products are cloned into suitable vectors for heterologous expression in e.g. Escherichia coli, and the protein fragment products so generated are screened for antigenicity using a suitable method, such as in 96-well plates of standard format, by adding recombinant fragments to individual wells. Sera from infected animals and from control animals are added, and the presence of host antibodies is detected by ELISA to identify a fragment according to the invention.

In another aspect, the present invention relates to a method for treatment or prevention of a disease caused by Treponema spp. comprising administering to a subject isolated protein having the amino acid sequence according to SEQ ID NO: 2, SEQ ID NO:4, SEQ ID NO:6, SEQ ID NO:18 or SEQ ID NO:20, or a fragment or derivative thereof capable of inducing an immune response to said protein. Said method may be used for a disease such as digital dermatitis. Fragments as described in more detail above are also useful in this aspect.

In a further aspect, the present invention relates to a pharmaceutical composition comprising an isolated protein having the amino acid sequence according to SEQ ID NO:2, SEQ ID NO:4, SEQ ID NO:6, SEQ ID NO:18 or SEQ ID NO:20, or a fragment or derivative thereof capable of inducing an immune response to said protein, and optionally pharmaceutically acceptable adjuvants, carriers and/or diluents.

Fragments as described in more detail above are also useful in this aspect.

In yet another aspect, the present invention relates to a method for detecting the presence of antibodies against proteins from Treponema spp. in a sample, comprising the steps:

Bringing said sample in contact with an isolated protein having the amino acid sequence according to SEQ ID NO:2, SEQ ID NO:4, SEQ ID NO:6, SEQ ID NO:18 or SEQ ID NO:20, or a fragment or derivative thereof capable of binding to antibodies produced by a subject in an immune response against said protein; and
Detecting antibodies binding to said protein, fragment or derivative.
Fragments according to the invention are also useful in this aspect.

Thus, as the skilled person appreciates from the above, a method according to the invention may be a simple test, which advantageously can be used to supplement and/or confirm clinical testing. A suitable format would e.g. be an indirect ELISA, which is a well known assay format readily available from commercial sources. As the skilled person will appreciate, other alternative formats using one or more proteins and/or protein fragments according to the invention are also embraced by the invention.

A further aspect of the invention relates to a method for in vitro diagnosis of a disease caused by Treponema spp. comprising the steps of:

Obtaining a sample of body fluid or tissue from a subject;
Bringing said sample in contact with an isolated protein having the amino acid sequence according to SEQ ID NO:2, SEQ ID NO:4, SEQ ID NO:6, SEQ ID NO:18 or SEQ ID NO:20 or a fragment or derivative thereof capable of binding to antibodies produced by a subject in an immune response against said protein; and
Detecting antibodies binding to said protein, fragment or derivative; wherein the presence of antibodies binding to said protein, fragment or derivative is indicative of a disease caused by Treponema spp.

Fragments as described in more detail above are also useful in this aspect.

Said method may be used for a disease such as digital dermatitis.

A further aspect of the invention relates to an antibody, or binding fragment thereof, binding specifically to an isolated protein having the amino acid sequence according to SEQ ID NO:2, SEQ ID NO:4, SEQ ID NO:6, SEQ ID NO:18 or SEQ ID NO:20.

Fragments as described in more detail above are also useful in this aspect.

Another aspect of the invention relates to a method for treatment or prevention of a disease caused by Treponema spp. comprising administering said antibody to a subject. Digital dermatitis is one example of such a disease.

A further aspect of the invention relates to a method for separation of Treponema bacteria from a sample, comprising the steps of:

Bringing said sample in contact with said antibody bound to a solid phase;
Allowing said antibody to bind to Treponema proteins in said Treponema bacteria; and
Separating said solid phase from said sample thereby separating said Treponema bacteria from said sample.

In said method, the separation may be achieved by for example immuno-magnetic separation.

Another aspect of the invention relates to a nucleic acid molecule encoding the protein, fragment or derivative according to the present invention. In one embodiment the nucleic acid molecule encoding the protein has a nucleotide sequence selected from the group consisting of SEQ ID NO: 1, SEQ ID NO:3, SEQ ID NO:5, SEQ ID NO:17, or SEQ ID NO:19 and parts thereof.

A further aspect of the invention relates to a vector comprising said nucleic acid molecule and optionally regulatory sequences for expression in a host cell.

Another aspect of the invention relates to a transgenic host cell comprising said vector.

The present invention also relates to a method for producing a protein, fragment or derivative according to the present invention, comprising the steps of:

culturing the host cell according to the present invention in a suitable medium; and
isolating said protein, fragment or derivative from said medium.

### EXPERIMENTAL PART

The present examples are provided for illustrative purposes only, and should not be construed as limiting the scope of the invention as defined by the appended claims.

Shotgun phage display was used to identify three immunogenic proteins in an isolate (V1) of the DD Treponema phylotype closely related to T. phagedenis. This phylotype has been indicated in several studies to be a key agent in the pathogenesis of DD (Klitgaard et al. 2008, Nordhoff et al. 2008, Yano et al. 2009). The phage library was selected against antibodies from a rabbit immunized with live bacteria.

A homolog to the well-characterized immunogenic protein TmpA of T. pallidum subsp. pallidum was identified as well as two proteins without homology to any known spirochetal protein. The complete amino acid sequence of these proteins was predicted from a genomic sequence of V1 generated by 454 Sequencing™. The three specific immunogenic proteins, and their amino acid sequences, are described in SEQ ID NO: 2, 4 and 6.

The proteins, fragments and derivatives according to the first aspect of the invention may be isolated from a culture of the Treponema phylotype closely related to T. phagedenis strain V1, or, preferably, recombinantly produced as described below.

Western blot has been performed to show that both antibodies from the immunized rabbit as well as naturally infected cattle bind to the recombinantly produced TmpA homolog and the Ttm fragment.

Pilot ELISA runs have been made and a difference in absorbance has been recorded between sera from cattle with and without DD (table 1). There were only a few overlaps between the results in the healthy and the infected group using single antigens (the TmpA homolog or the Ttm fragment) and no overlaps using a combination of the two antigens.

The immunogenic proteins according to the present invention, and active fragments thereof, can be used in vaccines against diseases caused at least in part by the Treponema phylotype closely related to T. phagedenis, or other Treponema spp.

In certain embodiments, the complete proteins are used in vaccines. In certain embodiments only fragments comprising the relevant epitopes are used. In certain embodiments one or more epitopes of one or more proteins are combined in a single molecule and used in a vaccine. The recombinant proteins, derivatives or fragments thereof may be thus used alone or in different combinations or as fusion proteins of the binding epitopes.

### Methods

Helper phage, bacterial strains, growth conditions, and DNA-techniques Phage R408 (Promega) was used as helper phage. Escherichia coli TG1 (Δ(lac-proAB) Δ(mcrB-hsdSM)5 (r_{K}⁻m_{K}⁻) thi-1 supE [F′traD36 proAB lacl^{q}ZΔM15]; Stratagene) was used as host in all experiments involving phages or phagemids and grown in Luria-Bertani broth (LB) or on Luria-Bertani agar (LA). When appropriate, 50 μg/ml ampicillin (C₁₆H₁₈N₃O₄SNa, Roche) was added. Incubations were at 37° C. Chromosomal DNA from Treponema sp. strain V1 was used for construction of the phage library. The Treponema strain was grown in flasks with FABGS (fastidious anaerobe broth, LAB 71, LabM, Lancashire, UK with 2.0 g D-glucose per liter and 25% fetal calf serum, S 0115, Biochrom AG, Germany) incubated at 37° C., in anaerobic jars on a shaker (90 rpm). Broth cultures were washed three times in isotonic saline (pH 6.3), followed by one wash in phosphate buffered saline (PBS, pH 7.3). The Treponema DNA was prepared by conventional phenol-chloroform extraction.

Restriction and modification enzymes were from MBI Fermentas AB and used according to manufacturer's instructions. Plasmids were prepared using QIAprep™ Miniprep (QIAGEN).

### Immunization and Purification of Polyclonal Antibodies

This part of the study was approved by the ethical committee on animal experiments in Uppsala (C 300/8). A New Zealand white rabbit was immunized subcutaneously with a live culture of Treponema sp. strain V1. A dose of approximately 10⁹ bacteria, washed twice and dispensed in a volume of 0.5 ml isotonic saline, was injected twice with 20 days in between. Serum from the final bleed at day 38 post first immunization was used for purification of antibodies (IgG) for this study. Ten ml serum was sterile filtered through a 0.45 μm syringe filter and applied to a 5 ml HiTrap™ Protein G HP column (GE Healthcare). Rabbit IgG was purified according to the manufacturer's instructions using the Ab Buffer Kit (GE Healthcare). Eluates of purified antibodies were desalted using Zeba Spin Desalting columns (Pierce) and stored in PBS at minus 20° C.

Construction of the Treponema sp. Phage Display Library and Selection of Binding Phages (Panning)

The phage library was constructed in the pG8SAET phagemid vector. Treponema sp. strain V1 chromosomal DNA was fragmented by sonication until the majority of the fragments were between 0.4-1.5 kb in length. The fragments were made blunt-ended by T4 DNA polymerase and T4 DNA kinase treatment and then ligated into SnaB1-digested and dephosphorylated phagemid vector pG8SAET using Ready-To-Go™. T4 DNA ligase tubes (GE Healthcare). The final library was generated by electrotransformation of the ligated material into E. coli TG1 cells (2.5 kV, 25 μF, 360Ω), infection with helper phage, and proliferation of phage particles. This procedure yielded 4.times.10.sup.7 transformants, considered as unique clones, 86% of which carried an insert, as determined by colony PCR on 14 randomly selected clones. The final library had a titer of 1.times.10.sup.11 colony forming units per ml. Phage displaying immunogenic polypeptides were isolated by panning against rabbit anti-Treponema sp. strain V1 IgG. Three panning experiments were performed. Microwells (MaxiSorp™, Nalge Nunc International) were coated with Zymed recombinant Protein G (Invitrogen) at a concentration of 10 μg in 200 μl 50 mM sodium carbonate, pH 9.5. Thereafter, the wells were blocked with phosphate buffered saline pH 7.4 with 0.05% Tween 20 (PBS-T). Rabbit anti-Treponema sp. strain V1 IgG was added at a concentration of 85 or 215 μg in 200 μl PBS or 100 μl PBS+100 μl crude E. coli lysate (for blocking). After washing, 200 μl of the phage library was added. The wells were incubated for 3 h at room temperature, or over night at 2° C., after which they were washed 25 times before phage were eluted by addition of 50 mM Na-citrate/140 mM NaCl pH 2.0. The eluate was immediately neutralised with 2 M Tris-buffer pH 8.0 and used to infect E. coli TG1, which were plated on LA-plates with ampicillin (LAamp). In one panning experiment, elution was also carried out by direct infection of TG1 cells added to the well by the bound phages. After incubation overnight, colonies were counted and 100 colonies transferred to an LAamp-plate. These colonies were then transferred to nitrocellulose-filters for screening of E-tag expression using mouse anti-Etag antibodies (GE Healthcare) and secondary horse radish peroxidase-labelled sheep anti-mouse antibodies (GE Healthcare). The remaining colonies were washed off the plates and superinfected with helper phage to make an enriched library/phage stock, which was used in the second enrichment cycle (repanning) according to the same protocol. In total, two repannings were performed. More than 200 E-tag positive colonies were chosen for plasmid preparation and sequence determination of the inserts using primer SAsekv (5′-TAT CTG GTG GCG TAA CAC CTG CT-3′). Plasmid DNA was sequenced on a 3730×1 DNA Analyzer (Applied Biosystems) at Uppsala Genome Centre and analyzed with the CLC Main Workbench software (CLC bio). Analyses of the inserts revealed nine, nine, and eight overlapping partial sequences, respectively, from three different genes.

### Genome Sequencing and Sequence Analysis

The chromosomal DNA of Treponema sp. strain V1 was sequenced and assembled at the KTH Genome Center at KTH Royal Institute of Technology, Stockholm, Sweden, using the Genome Sequencer FLX System, with long-read GS FLX Titanium chemistry and the 454 de novo assembler, Newbler (454 Life Sciences, Branford, Conn., USA). An additional De Novo assembly of the reads was made with CLC Genomics Workbench 3 (CLC bio) and for further sequence editing CLC Main Workbench 5 (CLC bio) was used.

The genome sequence was used to predict the full open reading frames and the corresponding amino acid sequences of the three immunogenic proteins. Homology searches were performed using the BLAST algorithm at the National Center for Biotechnology Information. The SignalP 3.0 Server with Gram-positive data was used for prediction of signal peptides. One protein was predicted as a lipoprotein according to Setubal et al. 2006. Genomic Treponema sp. strain V1 DNA for PCR was prepared with the DNeasy Blood & Tissue Kit (QIAGEN) following the protocol for Gram-negative bacteria. A 50 μl reaction mixture of 5 μl 10×Pfu Buffer with MgSO₄ (Fermentas), 0.2 mM of each deoxynucleotide, 0.2 μM of forward and reverse primer as indicated in Table 2, 1.25 U Pfu DNA polymerase (Fermentas) and 50 ng genomic DNA, was prepared. The thermal cycling conditions were 95° C. for 1 min, 30 cycles of 95° C. for 30 s, 50° C. for 30 s, and 72° C. for 3 min and a final extension at 72° C. for 5 min. PCR products were analyzed by agarose gel electrophoresis and purified with the illustra GFX PCR DNA and Gel Band Purification Kit (GE Healthcare). Purified amplicons were digested with either BamHI and XhoI or NdeI and SapI according to the manufacturer's instructions (Fast digest, Fermentas) and purified as described earlier. The digested amplicons were ligated into the respective vector—BamHI and XhoI digested pGEX-6P-1 (bulk GST purification module, GE Healthcare) or NdeI and SapI digested pTXB 1 (IMPACT™ Kit, New England BioLabs)—using the ReadyToGo T4DNA Ligase (GE Healthcare). Ligated material were electrotransformed into competent Escherichia coli strain BL21(DE3) (GST) or ER2566 (IMPACT) and spread on LA supplemented with ampicillin (final conc. 50 μg/ml). The presence of inserts in a number of colonies was analyzed by PCR using the vector sequencing primers. Clones with a correct size insert were further analyzed by DNA sequencing.

### Production of Recombinant Immunogenic Treponema Proteins

Commercially available protein expression and purification systems such as the bulk GST purification module (GE Healthcare) or the IMPACT™ Kit (New England BioLabs) were used for production of recombinant immunogenic Treponema proteins according to manufacturer's instructions. Recombinant clones were grown at 37° C. in LB media supplemented with ampicillin (final conc. 50 μg/ml). At an optical density (OD₆₀₀.) ˜0.6, the growth medium was supplemented with IPTG (final konc. 0.3 mM) and the growth temperature shifted to 20° C. After incubation over night the cells were harvested and resuspended in a buffer [20 mM Tris-HCl (pH 8.0), 500 mM NaCl, 0.1 mM EDTA, and 0.05% (v/v) TWEEN20] and lysed by freezing and thawing. After centrifugation, the supernatants were sterile filtrated and applied onto a chitin column. The columns were washed extensively using the same buffer and treated subsequently with cleavage buffer [20 mM Tris-HCl (pH 8.0), 50 mM NaCl, 0.1 mM EDTA, and 30 mM dithiothreitol (DTT)]. The eluted samples containing the antigens were dialysed against phosphate-buffered saline [PBS; 137 mM NaCl, 2.7 mM KCl, 10 mM Na₂HPO₄, 1.4 mM KH₂PO₄ (pH 7.4)].

In the GST-glutathione affinity system, according to the procedure described above, after growth, induction and harvest, the E. coli cells were suspended in PBS supplemented with TWEEN20, final conc. 0.1% (v/v) (PBST) whereupon the cells were lysed by freezing and thawing. After centrifugation, the supernatant was sterile filtrated and batch purified with Glutathione-sepharose beads. After extensive washing using PBST the fusion protein was eluted with glutathione elution buffer or treated with scissor protease to release the produced protein. Finally, the amounts of antigens obtained were determined using spectrophotometry and the quality analyzed by SDS-PAGE coomassie staining. The proteins were stored finally at −20° C.

### Enzyme-Linked Immunosorbant Assays (ELISAs)

Advantages with using purified proteins recombinantly produced in E. coli compared to whole cell lysates in an ELISA are the possibilities of large-scale production and optimization of the test (different combinations of proteins, often less background due to higher specificity). Initial ELISA tests were performed with sera from cattle with and without digital dermatitis (DD) and with sera from the immunized rabbit (above). Microplates (PolySorp™ Nalge Nunc International) were coated with recombinant TmpA homolog and/or recombinant tail tape measure protein fragment at a concentration of 2.5 μg and/or 0.31 μg, respectively, in 100 μl 50 mM sodium carbonate, pH 9.5, overnight at 2° C. Wells were washed twice with 400 μl phosphate buffered saline pH 7.4 with 0.05% Tween 20 (PBS-T). Thereafter, the wells were blocked with PBS-T for one hour at room temperature. One hundred μl serum or PBS-T was added to each well. Four dilutions of each serum were used −1:25, 1:50, 1:100, and 1:200. The microplates were incubated at 37° C. for one hour and then washed three times with 400 μl PBS-T. Horse-radish peroxidase conjugated swine-anti rabbit (Dako) and rabbit-anti cow (Dako) antibodies were added to the relevant wells, diluted 1:4000 and 1:500, respectively, and plates were incubated for one hour at 37° C. The wells were washed three times with 400 μl PBS-T after which 100 μl solution consisting of 20 mM tetramethylbenzidine (TMB) mixed 1:20 with 0.1 M potassium citrate/H₂O₂ (230 n1/1) pH 4.25, was added. The plates were incubated for 10 minutes at room temperature. To stop the reaction 50 μl 10% sulfuric acid was added. Optical density (OD) was measured at 450 nm and the readings were corrected against a sample buffer blank.

### Immunomagnetic Separation Using Antibodies

Treponema spp. are fastidious organisms that require complex culture media and anaerobic environments for growth. Additionally, samples from cattle claws have a plethora of other bacteria contaminating the cultures. An option to concentrate and purify Treponema spp. for culturing and DNA isolation is immunomagnetic separation (Demirkan et al. 1999, Demirkan et al. 2001, Choi et al. 1996) Immunomagnetic beads covalently coated with for example anti-rabbit IgG coupled with rabbit antibodies raised against said immunogenic proteins, derivatives or active fragments thereof can be used for specific separation of the DD Treponema phylotype closely related to T. phagedenis.

### Vaccines

Bacterial proteins stimulating the immune system to antibody production can also be used for vaccine development. Recombinant proteins can be combined with immune-stimulating complexes (ISCOMs) and/or whole cell lysates to increase the immune response in the animal and hence the protection against the disease-causing agent/s.

### Further Experiments Performed

### Enzyme-Linked Immunosorbant Assay (ELISA)

Materials and methods: The assays were performed with sera from eight dairy cows with acute DD from the herd from which Tp1 strain V1 was isolated, two cows from another herd with no known history of DD, and five calves 6-7 months of age. Digital dermatitis diagnosis was made by visual examination. Microplates (PolySorp™, Nalge Nunc International) were coated with recombinant proteins at concentrations of 1 mg/ml TmpA, 0.8 mg/ml Ttm or 0.02 μg/mlPrrA in 100 μl 50 mM sodium carbonate, pH 9.5, overnight at 2° C. Wells were washed twice with PBS-T and blocked with PBS-T for one hour at room temperature. 171 One hundred μl serum diluted 1:100 in PBS-T was added to each well. The microplates were incubated at 37° C. for one hour and then washed with PBS-T. Horse-radish peroxidase (HRP) conjugated rabbit anti-bovine IgG antibodies (Sigma) diluted 1:8000 or monoclonal 22:26 anti-bovine IgG-HRP antibodies (Svanova Biotech AB) diluted 1:4000 were added to the wells and plates were incubated for one hour at 37° C. The wells were washed three times with PBS-T after which 100 μl solution consisting of 1 mM tetramethylbenzidine and 0.006% H2O2 in 0.1 M potassium citrate pH 4.25, was added. The plates were incubated for 10 minutes at room temperature. To stop the reaction 50 μl 10% sulfuric acid was added. Optical density was measured at 450 nm and the readings were subtracted by the optical density of a sample buffer blank giving the corrected optical density (COD).

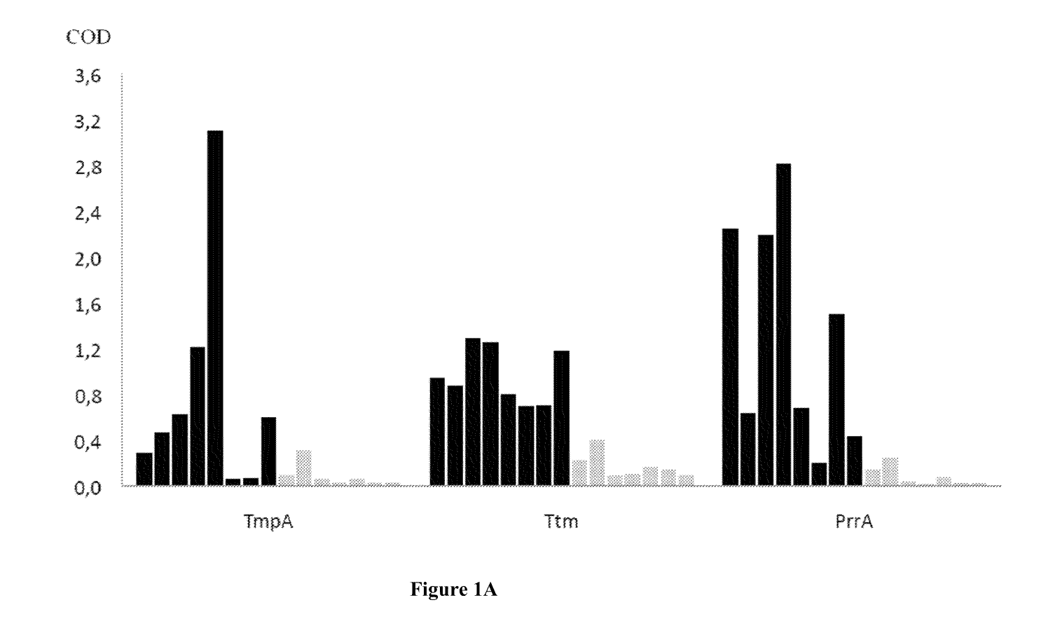

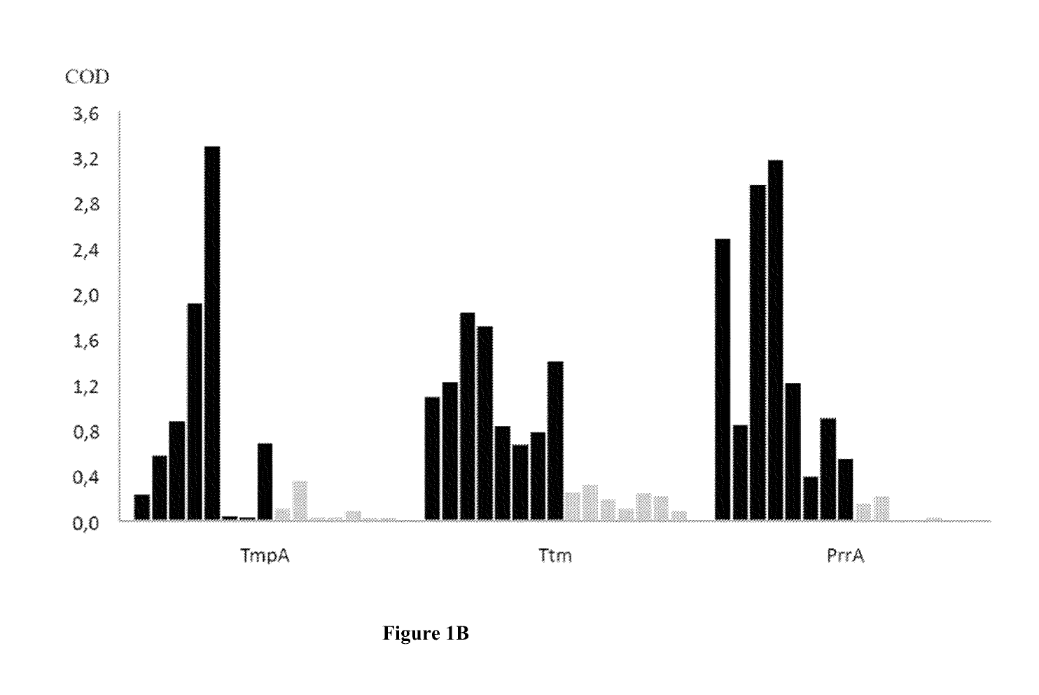

Results: The three immunogenic proteins identified in Tp1 strain V1 were produced recombinantly; PrrA as a full-length mature protein from aa+1 relative the cysteine residue of the predicted lipoprotein signal peptide to the last aa before the stop codon (aa:s 22-251), TmpA from aa+7 relative the cystein residue of the predicted lipoprotein signal peptide to the last aa before the stop codon (aa:s 29-344), and Ttm as a partial polypeptide covering aa:s 689-970, which are the aa:s constituting the consensus sequence of the overlapping Ttm sequences from the panning experiments. These proteins/polypeptides were used as antigens in indirect ELISAs where serum samples from cattle with and without DD were analyzed for presence of antibodies against the antigens. Different concentrations of antigen, sera, and secondary antibody were tested in pilot experiments (data not shown). The conditions under which the best discrimination between cattle with and without DD was achieved were used in the final experiment. For the TmpA antigen, the optical density for three samples from cattle with DD was lower than the highest value for the samples from clinically healthy 269 cattle, while the tests with Ttm and PrrA were discriminatory in all cases but one (FIGS. 1a) and b)).

TABLE 1
Initial ELISA tests of sera from cattle with and without digital dermatitis (DD). Optical density (OD) was measured at 450 nm
and the readings were corrected against a sample buffer blank. Sera were diluted 1:100, the concentration of TmpA was
2.5 μg/well and of the putative tail tape measure protein fragment 0.31 μg/well.

| | Cattle without DD (OD, 450 nm) | | | | | | | Cattle with DD (OD, 450 nm) | | | | | | | | |
|---|---|---|---|---|---|---|---|---|---|---|---|---|---|---|---|---|
| Protein/protein fragment | 1670 | 1680 | 242 | 243 | 245 | 246 | 251 | 5510 | RK | SK | 413 | 571 | 1004 | 2326 | 5542 | 6437 |
| TmpA homolog | 1.23 | 1.06 | 1.28 | 0.91 | 1.31 | 0.75 | 0.73 | 2.55 | 1.25 | 1.7 | 2.18 | 2.26 | 2.44 | 1.82 | 2.96 | 3.15 |
| Putative tail tape measure protein | 1.30 | 1.47 | 1.08 | 1.20 | 1.59 | 0.75 | 0.70 | 2.14 | 1.66 | 1.91 | 1.61 | 1.86 | 2.06 | 1.76 | 1.74 | 1.56 |
| fragment | | | | | | | | | | | | | | | | |
| TmpA homolog + putative tail | 1.36 | 1.23 | 0.63 | 0.90 | 0.91 | 0.83 | 0.60 | 2.47 | 1.83 | 1.84 | 1.40 | 1.73 | 2.17 | 1.87 | 2.65 | 2.03 |
| tape measure protein fragment | | | | | | | | | | | | | | | | |

TABLE 2
Deoxyoligoribonucleotides

| SEQ ID | Name of forward (fwd) and | Sequence, 5′ to 3′ of each primer. Restriction |
|---|---|---|
| NO | reverse (rev) primer pair | enzyme cleavage sites are indicated in bold. |
| 7 | GSTtmpAF1 (fwd) | GGT GGT GGA TCC AAA GCG GAA CAA GAA GCT CA |
| 8 | GSTtmpAR1 (rev) | GGT GGT CTC GAG TCA TTG TAC ACC TCC CTC TA |
| 9 | GSTkallaF1 (fwd) | GGT GGT GGA TCC AAG AAA GAG CTG TTA GAT TT |
| 10 | GSTkallaR1 (rev) | GGT GGT CTC GAG TTA TTT ATC AAT TTC TGC CAA |
| 11 | GSTPGKEEF1 (fwd) | GGT GGT GGA TCC CAA GGT CCA GCT AAC CCC ACA |
| 12 | GSTPGKEER1 (rev) | GGT GGT CTC GAG TTA GAG CTT CTC TAG CAC AAA |
| 13 | ImpactPGKEEF1 (fwd) | GGT GGT CAT ATG CAA GGT CCA GCT AAC CCC ACA |
| 14 | ImpactPGKEER1 (rev) | GGT GGT TGC TCT TCC GCA GAG CTT CTC TAG CAC AAA |
| 15 | ImpactkallaF1 (fwd) | GGT GGT CAT ATG AAG AAA GAG CTG TTA GAT TT |
| 16 | ImpactkallaR1 (rev) | GGT GGT TGC TCT TCC GCA TTT ATC AAT TTC TGC CAA |

### Identification of Additional Proteins

More specifically, the promoter region of the protein of SEQ ID NO 6, known as PrrA, comprises a −10 box and a −35 box with sequences identical to the consensus sequences of these elements. Between the −10 box and the −35 box there are a number of TA repeats. Persson et al (Persson A, Jacobsson K, Frykberg L, Johansson K E, Poumarat F. 2002. Variable surface protein Vmm of Mycoplasma mycoides subsp. mycoides small colony type. J. Bacteriol. 184:3712-22) have identified such promoter sequences in other bacteria, e.g. Mycoplasma mycoides subsp. mycoides and they were shown to regulate the expression of the gene in a phase variable manner. Phase variation refers to a reversible switch between an “all-or-none” (on/off) expressing phase, resulting in variation in the level of expression of one or more proteins between individual cells of a clonal population. The molecular basis for phase varaiation of one M. mycoides subsp. mycoides protein, Vmm, was shown to be a variation of the number of TA repeats in the promoter spacer that separates the −35 box from the −10 box. An active promoter was shown to have a spacer of 10 TA repeats while any other number of repeats was found to disrupt the functionality of the promoter. The length of the spacer is determined by dinucleotide insertion or deletion mutations.

Another feature of PrrA common with phase variable proteins in e.g. Mycoplasma bovis (Lysnyansky et al. 1999) is the presence of regions composed of reiterated coding sequences that create a periodic polypeptide structure. The variable membrane surface lipoproteins (Vsps) of M. bovis are subject to size variation due to spontaneous insertions or deletions of these periodic units.

The M. mycoides subsp. mycoides phase and antigenic variable proteins were shown to be immunogenic in cattle.

These three common features for PrrA and the Mycoplasma proteins (promoter sequences, periodic amino acid repeat sequences, immunogenicity)—and the fact that outer membrane antigenic variation has been demonstrated for other spirochetes (Borrelia (Zhang et al. 1997) and Brachyspira (McCaman et al. 2003))—inspired us to search the Treponema phagedenis-like strain V1 genome for other proteins regulated the same way, i.e. with similar promoter sequences. Another two proteins, Vps (variable promoter spacer) protein A and VpsB were identified. These proteins have similar promoter sequences and identical (VpsB) or similar (VpsA) signal peptides as PrrA.

The presence of PrrA (SEQ ID NO:1), VpsA (SEQ ID NO:18) and VpsB (SEQ ID NO:20) has been investigated by PCR in 11 T. phagedenis-like isolates. The gene for VpsA was present in all investigated isolates, PrrA in 8 isolates and VpsB in 9 isolates. Not one isolate lacked both the PrrA gene and the VpsB gene. The size of the PCR products for PrrA and VpsB varied why some PCR products were sequenced, 4 for PrrA and 4 (including V1) for VpsB. Different numbers of amino acid repeat units were detected in all PCR products except for one VpsB-PCR product that was identical to the one from V1.

Presence of antibodies in serum from naturally infected cattle with clinical DD (14 herds) and from cattle with no clinical signs of DD (10 herds) has been investigated (Table 3) through indirect ELISA. This study included Ttm (SEQ ID NO:4), PrrA (SEQ ID NO:6), VpsA (SEQ ID NO:18) and VpsB (SEQ ID NO:20).

TABLE 3
Number of test positive cows per herd, by antigen. The cut-offs used are based on the median test results
(plus two standard deviations) of the cows from the herds where DD has not been clinically detected.

| | | | | | Number of positive cows | | | | |
|---|---|---|---|---|---|---|---|---|---|
| | | Number of cows | | | PrrA | | | Ttm | |
| | | Cows | Cows | Total | Cows | Cows | Total | Cows | Cows |
| | | with | without | number | with | without | number | with | without |
| | | clinical | clinical | of | clinical | clinical | of | clinical | clinical |
| Herd | | DD | signs | cows | DD | signs | cows | DD | signs |
| 1 | DD free | 0 | 10 | 10 | 0 | 0 | 0 | 0 | 0 |
| 2 | DD free | 0 | 10 | 10 | 0 | 0 | 0 | 0 | 0 |
| 3 | DD free | 0 | 10 | 10 | 0 | 3 | 3 | 0 | 0 |
| 4 | DD free | 0 | 10 | 10 | 0 | 1 | 1 | 0 | 0 |
| 5 | DD free | 0 | 10 | 10 | 0 | 1 | 1 | 0 | 0 |
| 6 | DD free | 0 | 10 | 10 | 0 | 0 | 0 | 0 | 6 |
| 7 | DD free | 0 | 10 | 10 | 0 | 0 | 0 | 0 | 0 |
| 8 | DD free | 0 | 10 | 10 | 0 | 0 | 0 | 0 | 1 |
| 9 | DD free | 0 | 9 | 9 | 0 | 0 | 0 | 0 | 0 |
| 10 | DD free | 0 | 10 | 10 | 0 | 0 | 0 | 0 | 0 |
| 11 | DD case | 8 | 10 | 18 | 7 | 4 | 11 | 1 | 3 |
| 12 | DD case | 4 | 8 | 12 | 3 | 3 | 6 | 0 | 1 |
| 13 | DD case | 5 | 13 | 18 | 0 | 0 | 0 | 2 | 1 |
| 14 | DD case | 8 | 9 | 17 | 6 | 4 | 10 | 0 | 0 |
| 15 | DD case | 10 | 10 | 20 | 0 | 0 | 0 | 0 | 0 |
| 16 | DD case | 21 | 13 | 34 | 2 | 0 | 2 | 0 | 0 |
| 17 | DD case | 11 | 10 | 21 | 3 | 1 | 4 | 0 | 0 |
| 18 | DD case | 12 | 10 | 22 | 0 | 1 | 1 | 0 | 0 |
| 19 | DD case | 11 | 11 | 22 | 2 | 0 | 2 | 1 | 2 |
| 20 | DD case | 14 | 10 | 24 | 8 | 2 | 10 | 1 | 0 |
| 21 | DD case | 9 | 5 | 14 | 4 | 1 | 5 | 2 | 0 |
| 22 | DD case | 8 | 6 | 14 | 6 | 2 | 8 | 0 | 1 |
| 23 | DD case | 6 | 10 | 16 | 0 | 0 | 0 | 0 | 2 |
| 24 | DD case | 6 | 11 | 17 | 5 | 3 | 8 | 0 | 3 |
| | | | Number of positive cows | | | | | | |
| | | | Ttm | VpsB | | | VpsA | | |
| | | | Total | Cows | Cows | Total | Cows | Cows | Total |
| | | | number | with | without | number | with | without | number |
| | | | of | clinical | clinical | of | clinical | clinical | of |
| | Herd | | cows | DD | signs | cows | DD | signs | cows |
| | 1 | DD free | 0 | 0 | 0 | 0 | 0 | 0 | 0 |
| | 2 | DD free | 0 | 0 | 0 | 0 | 0 | 1 | 1 |
| | 3 | DD free | 0 | 0 | 1 | 1 | 0 | 1 | 1 |
| | 4 | DD free | 0 | 0 | 1 | 1 | 0 | 0 | 0 |
| | 5 | DD free | 0 | 0 | 0 | 0 | 0 | 0 | 0 |
| | 6 | DD free | 6 | 0 | 2 | 2 | 0 | 4 | 4 |
| | 7 | DD free | 0 | 0 | 0 | 0 | 0 | 0 | 0 |
| | 8 | DD free | 1 | 0 | 1 | 1 | 0 | 0 | 0 |
| | 9 | DD free | 0 | 0 | 0 | 0 | 0 | 0 | 0 |
| | 10 | DD free | 0 | 0 | 1 | 1 | 0 | 0 | 0 |
| | 11 | DD case | 4 | 4 | 5 | 9 | 3 | 3 | 6 |
| | 12 | DD case | 1 | 0 | 0 | 0 | 1 | 3 | 4 |
| | 13 | DD case | 3 | 2 | 1 | 3 | 3 | 2 | 5 |
| | 14 | DD case | 0 | 7 | 3 | 10 | 6 | 5 | 11 |
| | 15 | DD case | 0 | 1 | 0 | 1 | 2 | 0 | 2 |
| | 16 | DD case | 0 | 3 | 1 | 4 | 7 | 1 | 8 |
| | 17 | DD case | 0 | 3 | 2 | 5 | 0 | 0 | 0 |
| | 18 | DD case | 0 | 6 | 1 | 7 | 2 | 1 | 3 |
| | 19 | DD case | 3 | 9 | 2 | 11 | 10 | 5 | 15 |
| | 20 | DD case | 1 | 5 | 1 | 6 | 0 | 0 | 0 |
| | 21 | DD case | 2 | 4 | 2 | 6 | 8 | 3 | 11 |
| | 22 | DD case | 1 | 4 | 2 | 6 | 5 | 1 | 6 |
| | 23 | DD case | 2 | 1 | 0 | 1 | 1 | 1 | 2 |
| | 24 | DD case | 3 | 4 | 3 | 7 | 1 | 1 | 2 |

### REFERENCES

Berry, S. L., Ertze, R. A., Read, D. H., Hird, D. W., 2004, Field evaluation of prophylactic and therapeutic effects of a vaccine against (Papillomatous) Digital Dermatitis of dairy cattle in two Californian dairies. In: Proceedings of the 13^{th} International Symposium and Conference on Lameness in Ruminants, Maribor, Slovenija, p. 147.
Blowey, R. W., Sharp, M. W., 1988. Digital dermatitis in dairy cattle. Vet. Rec. 122, 505-508.
Choi, B. K., Wyss, C., Gobel, U. B., 1996, Phylogenetic analysis of pathogen-related oral spirochetes. J Clin Microbiol. 34, 1922-1925.
Choi, B.K., Nattermann, H., Grund, S., Haider, W., Gobel, U. B., 1997. Spirochetes from digital dermatitis lesions in cattle are closely related to treponemes associated with human periodontitis. Int. J. Syst. Bacteriol. 47, 175-181.
Demirkan, I., Carter, S. D., Hart, C. A., Woodward, M. J., 1999. Isolation and cultivation of a spirochaete from bovine digital dermatitis. Vet. Rec. 145, 497-498.
Demirkan, I., Walker, R. L., Murray, R. D., Blowey, R. W., Carter, S. D., 1999, Serological evidence of spirochaetal infections associated with digital dermatitis in dairy cattle. Vet J. 157, 69-77.
Demirkan, I., Carter, S. D., Winstanley, C., Bruce, K. D., McNair, N. M., Woodside, M., Hart, C. A., 2001, Isolation and characterisation of a novel spirochaete from severe virulent ovine foot rot. J Med. Microbiol. 50, 1061-1068.
Evans, N. J., Brown, J. M., Demirkan, I., Murray, R. D., Vink, W. D., Blowey, R. W., Hart, C. A., Carter, S. D., 2008. Three unique groups of spirochetes isolated from digital dermatitis lesions in UK cattle. Vet. Microbiol. 30, 141-50.
Gupta, R. B., Fincher, M. G., Bruner, D. W., 1964. A study of the etiology of foot-rot in cattle. Cornell Vet. 54, 66-77.
Hillström, A., Bergsten, C., 2005. Digital dermatitis—a new infectious foot disease in Swedish dairy cattle. Svensk Vet. Tidn. 57, 15-20.
Keil, D. J., Liem, A., Stine, D. L., Anderson, G. A., 2002, Serological and clinical response of cattle to farm specific digital dermatitis bacterins. In: Proceedings of the 12^{th} International Symposium on Lameness in Ruminants, Orlando, Fla., USA, p. 385.
Klitgaard, K., Boye, M., Capion, N., Jensen, T. K., 2008, Evidence of multiple Treponema phylotypes involved in bovine digital dermatitis as shown by 16S rRNA gene analysis and fluorescence in situ hybridization. J Clin Microbiol. 46, 3012-3020. Epub 2008 June 3018.
Losinger W. C., 2006. Economic impacts of reduced milk production associated with papillomatous digital dermatitis in dairy cows in the USA. J. Dairy Res. 73, 244-256.
Manske, T., Hultgren, J., Bergsten, C., 2002. Topical treatment of digital dermatitis associated with severe heel-horn erosion in a Swedish dairy herd. Prey. Vet. Med. 53, 215-231.
Moter, A., Leist, G., Rudolph, R., Schrank, K., Choi, B. K., Wagner, M., Gael, U. B., 1998. Fluorescence in situ hybridization shows spatial distribution of as yet uncultured treponemes in biopsies from digital dermatitis lesions. Microbiology 144, 2459-2467.
Nordhoff, M., Moter, A., Schrank, K., Wieler, L. H., 2008, High prevalence of treponemes in bovine digital dermatitis-a molecular epidemiology. Vet Microbiol. 131, 293-300. Epub 2008 April 2022.
Read, D., Walker, R., 1996. Experimental transmission of papillomatous digital dermatitis (footwarts) in cattle. Vet. pathol. 33, 607.
Read, D., Nordhausen, R., Walker, R. L., 1998. Pathogenesis of experimental papillomatous digital dermatitis (footwarts) in cattle: Bacterial morphotypes associated with early lesion development. In: Lischer C. and Ossent P. (Eds.), Proceedings 10th international symposium on lameness i ruminants, Lucerne, Switzerland, p. 271.
Rodriguez-Lainz, A., Hird, D. W., Carpenter, T. E., Read, D. H., 1996. Case-control study of papillomatous digital dermatitis in Southern California dairy farms. Prey. Vet. Med. 28, 117-131.
Setubal, J. C., Reis, M., Matsunaga, J., Haake, D. A., 2006, Lipoprotein computational prediction in spirochaetal genomes. Microbiol. 152, 113-121.
Trott, D. J., Moeller, M. R., Zuerner, R. L., Goff, J. P., Waters, W. R., Alt, D. P., Walker, R. L., Wannemuehler, M. J., 2003, Characterization of Treponema phageden is-like spirochetes isolated from papillomatous digital dermatitis lesions in dairy cattle. J Clin Microbiol. 41, 2522-2529.
Vink, W. D., Jones, G., Johnson, W. O., Brown, J., Demirkan, I., Carter, S. D., French, N. P., 2009, Diagnostic assessment without cut-offs: application of serology for the modelling of bovine digital dermatitis infection. Prey Vet Med. 92, 235-248.
Walker, R. L., Read, D. H., Loretz, K. J., Nordhausen, R. W., 1995. Spirochetes isolated from dairy cattle with papillomatous digital dermatitis and interdigital dermatitis. Vet. Microbiol. 47, 343-355.
Walker, R. L., Read, D. H., Loretz, K. J., Hird, D. W., Berry, S. L., 1997, Humoral response of dairy cattle to spirochetes isolated from papillomatous digital dermatitis lesions. Am J Vet Res. 58, 744-748.
Yano, T., Moe, K. K., Yamazaki, K., Ooka, T., Hayashi, T., Misawa, N., 2009, Identification of candidate pathogens of papillomatous digital dermatitis in dairy cattle from quantitative 16S rRNA clonal analysis. Vet Microbiol. 23, 23.
Hamsten C, Westberg J, Bolske G, Ayling R, Uhlén M, Persson A. 2008. Expression and immunogenicity of six putative variable surface proteins in Mycoplasma mycoides subsp. mycoides SC. Microbiology. 154:539-49.
Lysnyansky I, Sachse K, Rosenbusch R, Levisohn S, Yogev D. 1999. The vsp locus of Mycoplasma bovis: gene organization and structural features. J. Bacteriol. 181:5734-41.
McCaman M T, Auer K, Foley W, Gabe J D. 2003. Brachyspira hyodysenteriae contains eight linked gene copies related to an expressed 39-kDa surface protein. Microbes Infect. 5:1-6.
Persson A, Jacobsson K, Frykberg L, Johansson K E, Poumarat F. 2002. Variable surface protein Vmm of Mycoplasma mycoides subsp. mycoides small colony type. J. Bacteriol. 184:3712-22)
Zhang J R, Hardham J M, Barbour A G, Norris S J. 1997. Antigenic variation in Lyme disease borreliae by promiscuous recombination of VMP-like sequence cassettes. Cell. 89:275-85.

1. An isolated protein having the amino acid sequence of SEQ ID NO:2, SEQ ID NO:4, SEQ ID NO:6, SEQ ID NO:18, or SEQ ID NO:20, or a fragment or derivative thereof capable of inducing an immune response to said protein or capable of binding to antibodies produced by a subject in an immune response against said protein.

2. The protein fragment according to claim 1, which comprises at least one sequence selected from the group consisting of residues 77-120 of SEQ ID NO:2; residues 922-948 of SEQ ID NO:4, or residues 689-909 of SEQ ID NO:4, residues 689-970 of SEQ ID NO:4, residues 66-83 of SEQ ID NO:6, and residues 163-183 of SEQ ID NO:6.

3. A method for treatment or prevention of a disease caused by Treponema spp., comprising administering to a subject the isolated protein of claim 1 or a fragment or derivative thereof capable of inducing an immune response to said protein.

4. The method according to claim 3, wherein a protein fragment which comprises at least one sequence selected from the group consisting of residues 77-120 of SEQ ID NO:2; residues 922-948 of SEQ ID NO:4, or residues 689-909 of SEQ ID NO:4, residues 689-970 of SEQ ID NO:4, residues 66-83 of SEQ ID NO:6, and residues 163-183 of SEQ ID NO:6 is administered.

5. The method according to claim 3, wherein the disease caused by Treponema spp. is digital dermatitis.

6. A pharmaceutical composition, comprising the isolated protein of claim 1 or a fragment or derivative thereof capable of inducing an immune response to said protein, and optionally one or more pharmaceutically acceptable adjuvant, carrier and/or diluent.

7. The composition according to claim 6, comprising a protein fragment which comprises at least one protein fragment comprising a sequence selected from the group consisting of residues 77-120 of SEQ ID NO:2; residues 922-948 of SEQ ID NO:4, residues 689-909 of SEQ ID NO:4, residues 66-83 of SEQ ID NO:6, and residues 163-183 of SEQ ID NO:6.

8. A method for detecting the presence of antibodies against proteins from Treponema spp. in a sample, comprising the steps of:
bringing said sample in contact with the isolated protein of claim 1 or a fragment or derivative thereof capable of binding to antibodies produced by a subject in an immune response against said protein; and
detecting antibodies binding to said protein, fragment or derivative.

9. The method according to claim 8, wherein a protein fragment which comprises at least one sequence selected from the group consisting of residues 77-120 of SEQ ID NO:2; residues 922-948 of SEQ ID NO:4, residues 689-909 of SEQ ID NO:4, residues 689-970 of SEQ ID NO:4, residues 66-83 of SEQ ID NO:6, and residues 163-183 of SEQ ID NO:6 is used in the step of contacting the sample with a fragment.

10. A method for in vitro diagnosis of a disease caused by Treponema spp., comprising the steps of:
obtaining a sample of body fluid or tissue from a subject;
bringing said sample in contact with the isolated protein of claim 1 or a fragment or derivative thereof capable of binding to antibodies produced by a subject in an immune response against said protein; and
detecting antibodies binding to said protein, fragment or derivative, wherein the presence of antibodies binding to said protein, fragment or derivative is indicative of a disease caused by Treponema spp.

11. The method according to claim 10, wherein a protein fragment which comprises at least one sequence selected from the group consisting of residues 77-120 of SEQ ID NO:2; residues 922-948 of SEQ ID NO:4, residues 689-909 of SEQ ID NO:4, residues 689-970 of SEQ ID NO:4, residues 66-83 of SEQ ID NO:6, and residues 163-183 of SEQ ID NO:6 is used in said step of contacting the sample with a fragment.

12. The method according to claim 10, wherein the disease is digital dermatitis.

13. An antibody, or an antigen binding fragment thereof, binding specifically to the isolated protein of claim 1.

14. The antibody according to claim 13 specifically binding to a protein fragment which comprises at least one sequence selected from the group consisting of residues 77-120 of SEQ ID NO:2; residues 922-948 of SEQ ID NO:4, residues 689-909 of SEQ ID NO:4, residues no 689-970 of SEQ ID NO:4, residues 66-83 of SEQ ID NO:6, and residues 163-183 of SEQ ID NO:6.

15. A method for treatment or prevention of a disease caused by Treponema spp., comprising administering to a subject the antibody according to claim 13.

16. The method according to claim 15, wherein the disease caused by Treponema spp. is digital dermatitis.

17. A method for separating Treponema bacteria from a sample, comprising the steps of:
bringing said sample into contact with the antibody according to claim 13 bound to a solid phase;
allowing said antibody to bind to a Treponema protein on said Treponema bacteria; and
separating said solid phase from said sample to thereby separate said Treponema bacteria from said sample.

18. The method according to claim 17, wherein said separating step is immunomagnetic separation.

19. A nucleic acid molecule encoding the protein, fragment or derivative of claim 1.

20. The nucleic acid molecule according to claim 19 having the nucleotide sequence of SEQ ID NO:1, SEQ ID NO:3, SEQ ID NO:5, SEQ ID NO:17, or SEQ ID NO:19.

21. A vector, comprising a nucleic acid molecule according to claim 19 and optionally regulatory sequences for expression in a host cell.

22. A transgenic host cell, comprising the vector according to claim 21.

23. A method for producing a protein, fragment or derivative according to of claim 1, comprising the steps of:
culturing a transgenic host cell comprising a vector which comprises a nucleic acid molecule encoding the protein, fragment or derivative of claim 1 in a suitable medium; and
isolating the protein, fragment or derivative from said medium.


SEQUENCE LISTING (20 sequences)

SEQ ID NO: 1 - DNA, 1050 bp, Treponema spp.
atggaggtcaatgagatgaaattaaaaactttggtttttagcttatccgcccttttccttgtattaggatttaccggttgtaaatctaaagcacaggcaaaagcggaacaagaagctcaagaacgaaaagcattgatggcggaaaatgctaaaatcgaaaaaagattgatgcaagccaaaaatgctgcaactgaagcggaagcaaatgtatattatcccgaaaagtttgcacaaattgaagatttggaaaaacaatcatcggaagcaaaagaacaggatgatttaaaaaaagcgaatagcttgggatctgctgccgccgacaaatacgagacgttggcgaataaaatgaagatagcgaatcaacgctcaaaaattgaagcaaataaacttgcaaaatatgacgaagaaagctatcgactcggggaagaggcggagaaaaagattgacggactttatgaaagcgattccgttgccgccttgcagacatcaaatgaaagccttatgtactataataaggtgatagatgcgggatataagtctctttcgcaagatgcaaaaaaaacagccgatgatgcaaaagcggcattaacggcggtaaaggttgccgcgagtttgaaaccccagcaagaggaagcggatggaatatatgctaaagctgaagaagcggagaattccgctcaatatgagcaatcatatggagggtacacttctgctgctcaagcatataacgatttaacacaaataattaaggcaaaacgattggaagctcaaaaggcaatgcaggcggcaaagacaaaacaagaactttccgcaaagcttgcaaatgaagcggataaagagagccctctacctgaaaatgccgaaggtttttcaaaagaaccgatagaagttgaacctctcccgacagatgtgttaaatgcacctcaagatgaaaaagctgaggaaacagttcccgttgaggaaatgaatgaaaattcttcggaagaagtaaacgggaatgcggaaaaaattgaatcgactgaagagccgatagagggaggtgtacaatga

SEQ ID NO: 2 - protein, 349 aa, Treponema spp.
MEVNEMKLKTLVFSLSALFLVLGFTGCKSKAQAKAEQEAQERKALMAENAKIEKRLMQAKNAATEAEANVYYPEKFAQIEDLEKQSSEAKEQDDLKKANSLGSAAADKYETLANKMKIANQRSKIEANKLAKYDEESYRLGEEAEKKIDGLYESDSVAALQTSNESLMYYNKVIDAGYKSLSQDAKKTADDAKAALTAVKVAASLKPQQEEADGIYAKAEEAENSAQYEQSYGGYTSAAQAYNDLTQIIKAKRLEAQKAMQAAKTKQELSAKLANEADKESPLPENAEGFSKEPIEVEPLPTDVLNAPQDEKAEETVPVEEMNENSSEEVNGNAEKIESTEEPIEGGVQ

SEQ ID NO: 3 - DNA, 3544 bp, Treponema spp.
atggcgaatgtatcaataggcaatttatatgcatccttaagtttaaaaagcgaaaagctgaaaagcggagcgattgaagctgaaggcgtcatgcgcaagcttgaaaaagacattgatgatataaaggacgctattaataataaacttgcgatgataggggctacattgtccgcaggcgtaacgctgccgcttactcttatgggaaagagtgcgcttgataccttctctcaatttgagcaagccatgcaaaatactttttccgttatgggagcgacggcttccgaaatggaaatgcttcgtaaaaaagcggaagacatgggagctgcgacccgatttagtgcaagccaagcggcagatgctctttatagtttaggctcggcaggacaaagcgcaaccgaagcggcagcaagtcttgacggtgtattaagcttagcaggagcgacggggagcgatttagcctttacttcagaaacaataacatcaacattatcacagtttaacatggaggcttcaaaagcgtcgcatgtcgcagatgtatacgcaaaggcaattagcaagagccaagcgaacatgacaaaactttcgtattcaatgcggtatgtcgggcctgttgcatccggacttgggataagtttagagacagcaacggcagctttaatgcgcctttacaataccggttacggcggcgagatggcaggtaactatcttaaaaacggattgcaaagattagcaagcggcggggaagacttcaaagcaaaacttgacgccatcgggcttagttatgacgaagtaaaccctaaaaccaacaaccttgccgacattatagaacgcttacgggagaagcaagtagacgtaacaaaggcgaatgaattattcggagatgcggcaggcggcgcaatgcttaagcttattgaaggcggaggggaagcaatacgaacaatggatgggttgctgcaatcttcacacggaacggcggaagaaatgcagaaaatgcaaaatacttcttttgcaaatacaaaagatgaattgtcatcggcgtttgaggcggtgcaaattacgcttacctctaatgtaataccggcggttgacatggtggcgaaggcatttactaaagtattacagtttgtcaacgagctgcccgttggggtgcaagtagcaggaacgggattagcgacaatggcggcagcggcgggacctttattgcttgttgccttgagtgtaaagaaaataaaggcggaaatggcagccctcaatgttacgatgatgtctaatccgatattccttatcggcacggcaattgcggcggggacggctttagcattaggagcgattgcacaaatacgaaaagcaaatgatgattatatacacagtgcaaaacggagtattgatgatattcggaaaatgcaagataacgcccttgctgaaggtaataaggggcggaagattaactcacttttggatgagtatgaaacgctcaagaataaaacaacaagaacggccgatgaacaagctcgatataatcaactgttggcagaattacaagagcttgttcctgatgttgtaacaaaactgaacgcgcaaggggaagcatttattgagaatgcggagaaagctcgagaggcggcacggcagcaattagaaacagagaaagcattaaacgatatggcacttgtaacgacaagagcaaaagcagaacatgccgaagcggttattgcaaaatacaaaaataaaccgaaagagctgcaagcgcagttaaaaattgaaacggaaaatgtaaaagcggcaacattactatatcagcagatacaagcaaaatacgaagagtttaaacatttgcaagaagcgggaagagaaaccgccgccgcaaaaataagagaagaaattcagcatctgtatctttcaaataacggggtacaagcggaaggcggggtatgggattatggcaatgtaaaaaaaatggtaaaagcgtttgaaaaacatcttaatacggttactggtaaacagcagaaaacgctgagattatatgagagcattaaaaacgctgtagaagaaaatattgcagcaaagaaagagctgttagatttagaagcaaggagccttgcaattgcaaaagctcaaggggaattatcagaagaagcaagcaaaaaagaaacagcaaaaacacgtcagcagcacgcagatacttcttgggacgaatatgagacaagaaaaaaacgatgggaaaaagaaaagaaagaagcggtctttttaggacgggaatttgatgaagttggtaagaaaatagaattcttgcaagcagaaataagcaatctgttacatcttaacgctgccgatgttgcagatggagtttttgcgctcaattcaaaagagcttaaaaaattaaagaaagagctggatacccttcttaagctacagggaaaaaataaaaagaatggcagcggcggcgaaaaatcaaaagatttgcaggcacacattgatgagcttgatagttactatcgcaagcgtatttcaatggcaaaagaatacggcttgaatgaagcaaaagaaacagaaaaatggttgaaagaacgaaaggcgctattagataaatacaataatgaaaaagaactccggaaaaaagaagcagggggaagcggtataactgttggagatgaaaaaaaacgaacagagtttttaggttatgccgattttacaaaatatcttaatgagcaaaaagaattacaaaaagaactggataaaacaagagaaaatatagaaaagacgaaagcattgcttgcgtcaaaagggaaagaccttaccccgaacgaaaggcgtgcagcaaaagattatttagaagacttacaagaaaaagcaaacaagttagagatagaactaaaacaatctcaatttacattggcagaaattgataaaactttagaagctattgagaattgtaacaagtctgattttgaactaaagcttattaatatcgaaaaagaaaaagagcgattactgcaagtcatagatgaggcaaagaaagcagggaaaataagcgaagaagaagcggaaaaagcgaagaaaaaagttcaagaaaaaacaaaaaaggacaagcaaggcgagtcagttaaaaatgctgatccatacgtaaagggagcaattggcattgcaaatgcgattgctgatgttatcgctgatgcgattgaaaaaggtggcgttgacggtcttaccgctatagcagcagcaggagagattatcggtcaaattggggatatggtcggaaactctgtggcaaaagctgtcttgggggcaacaagtgcggtagtcggaataactacaaaaattatgggcgcaatcaggcgaaagaatgctgcatattcagcagaacaaagagagaaaatgaaaaattttaatcagaaagtacaagaaggaatagaaaaaaatatccgcagagcaagtgaaatcgcaggggacgtgcctaaaacactggcaaaagcttttagcggaaaaaaactgtcaatcgatagtatttttgatagtcaatcagtggaactacaaaagaaaaaat

SEQ ID NO: 4 - protein, 1181 aa, Treponema spp.
MANVSIGNLYASLSLKSEKLKSGAIEAEGVMRKLEKDIDDIKDAINNKLAMIGATLSAGVTLPLTLMGKSALDTFSQFEQAMQNTFSVMGATASEMEMLRKKAEDMGAATRFSASQAADALYSLGSAGQSATEAAASLDGVLSLAGATGSDLAFTSETITSTLSQFNMEASKASHVADVYAKAISKSQANMTKLSYSMRYVGPVASGLGISLETATAALMRLYNTGYGGEMAGNYLKNGLQRLASGGEDFKAKLDAIGLSYDEVNPKTNNLADIIERLREKQVDVTKANELFGDAAGGAMLKLIEGGGEAIRTMDGLLQSSHGTAEEMQKMQNTSFANTKDELSSAFEAVQITLTSNVIPAVDMVAKAFTKVLQFVNELPVGVQVAGTGLATMAAAAGPLLLVALSVKKIKAEMAALNVTMMSNPIFLIGTAIAAGTALALGAIAQIRKANDDYIHSAKRSIDDIRKMQDNALAEGNKGRKINSLLDEYETLKNKTTRTADEQARYNQLLAELQELVPDVVTKLNAQGEAFIENAEKAREAARQQLETEKALNDMALVTTRAKAEHAEAVIAKYKNKPKELQAQLKIETENVKAATLLYQQIQAKYEEFKHLQEAGRETAAAKIREEIQHLYLSNNGVQAEGGVWDYGNVKKMVKAFEKHLNTVTGKQQKTLRLYESIKNAVEENIAAKKELLDLEARSLAIAKAQGELSEEASKKETAKTRQQHADTSWDEYETRKKRWEKEKKEAVFLGREFDEVGKKIEFLQAEISNLLHLNAADVADGVFALNSKELKKLKKELDTLLKLQGKNKKNGSGGEKSKDLQAHIDELDSYYRKRISMAKEYGLNEAKETEKWLKERKALLDKYNNEKELRKKEAGGSGITVGDEKKRTEFLGYADFTKYLNEQKELQKELDKTRENIEKTKALLASKGKDLTPNERRAAKDYLEDLQEKANKLEIELKQSQFTLAEIDKTLEAIENCNKSDFELKLINIEKEKERLLQVIDEAKKAGKISEEEAEKAKKKVQEKTKKDKQGESVKNADPYVKGAIGIANAIADVIADAIEKGGVDGLTAIAAAGEIIGQIGDMVGNSVAKAVLGATSAVVGITTKIMGAIRRKNAAYSAEQREKMKNFNQKVQEGIEKNIRRASEIAGDVPKTLAKAFSGKKLSIDSIFDSQSVELQKKK

SEQ ID NO: 5 - DNA, 756 bp, Treponema spp.
atgaaaaagttacgttttattagctttgccatattggcaacagcattgattgttgtatcttgccaaggtccagctaaccccacaaaagaacctggcaatgttcagaaagctgaggagaaaaaaccagaggtgaaagatccaaaagctgaggagaaaaaaccagaggtgaaagatccaaaagctgaggagaaaaaaccagaggagacatctccaggtacagaaaaaccagtagctccaggtaaagaagagccaggtaaagaagagccaggtaaagaagagccaggtaaagaagagccaggtaaagaagagccaggtaaagaagagccaggtaaagaagagcttcatgagtttattggcgagctttgtgaaatagcggaagaggaagaaatgcctgctgacgaagttagtcgttgggttgaactagcgcagaaggattttgaacaattcggcgtcaaaattgtggacaaagaagctgatcagccaattaaaaagggtgccagtgaagaagatcttaaaaaacgctttgtgctagagaaagatccaaatgctgagcttcatgggtttattggcgagctttgtgaaatagcggaagaggaagaaatgcctgctgacgaagttagtcgttgggttgaactagcgcaggaggattttgaacaattcggcgtcaaaattgtggacaaagaagctgatcagccaattaaaaagggtgccagtgaagaagatcttaaaaaacgctttgtgctagagaagctctaa

SEQ ID NO: 6 - protein, 251 aa, Treponema spp.
MKKLRFISFAILATALIVVSCQGPANPTKEPGNVQKAEEKKPEVKDPKAEEKKPEVKDPKAEEKKPEETSPGTEKPVAPGKEEPGKEEPGKEEPGKEEPGKEEPGKEEPGKEELHEFIGELCEIAEEEEMPADEVSRWVELAQKDFEQFGVKIVDKEADQPIKKGASEEDLKKRFVLEKDPNAELHGFIGELCEIAEEEEMPADEVSRWVELAQEDFEQFGVKIVDKEADQPIKKGASEEDLKKRFVLEKL

SEQ ID NO: 7 - DNA, 32 bp, Artificial Sequence
  Synthetic
ggtggtggatccaaagcggaacaagaagctca

SEQ ID NO: 8 - DNA, 32 bp, Artificial Sequence
  Synthetic
ggtggtctcgagtcattgtacacctccctcta

SEQ ID NO: 9 - DNA, 32 bp, Artificial Sequence
  Synthetic
ggtggtggatccaagaaagagctgttagattt

SEQ ID NO: 10 - DNA, 33 bp, Artificial Sequence
  Primer
ggtggtctcgagttatttatcaatttctgccaa

SEQ ID NO: 11 - DNA, 33 bp, Artificial Sequence
  Synthetic
ggtggtggatcccaaggtccagctaaccccaca

SEQ ID NO: 12 - DNA, 33 bp, Artificial Sequence
  Synthetic
ggtggtctcgagttagagcttctctagcacaaa

SEQ ID NO: 13 - DNA, 33 bp, Artificial Sequence
  Synthetic
ggtggtcatatgcaaggtccagctaaccccaca

SEQ ID NO: 14 - DNA, 36 bp, Artificial Sequence
  Synthetic
ggtggttgctcttccgcagagcttctctagcacaaa

SEQ ID NO: 15 - DNA, 32 bp, Artificial Sequence
  Synthetic
ggtggtcatatgaagaaagagctgttagattt

SEQ ID NO: 16 - DNA, 36 bp, Artificial Sequence
  Synthetic
ggtggttgctcttccgcatttatcaatttctgccaa

SEQ ID NO: 17 - DNA, 713 bp, Treponema spp.
  -35_signal (11)..(16)
  -10_signal (33)..(38)
caaaaaccacttgacatatatatatatatatatataatagatagtaactttttattacaggaggattttttatgaaaaagttacgttttcttggctttgccatagtggcaatagcattgattgtatcttgccctgggacaagtgccaccccaaaagcatctgcaaacactggtggtaaggttcaaccagctccacaaccagctccagctccaggtgaaagcggtaacacatcccctgatggaaatacaccactaggcggaacgaacgacctctccactgaggatgtgaaaaaggtagttgataaaatcaatcaaatgttcgaaggtttaggcgtagaagttacagacagtagccctgatgccccaatcactaaagagaccactccaaaccaacttaaagaacgatttaaatttaccaaaaaccctgctttagccgatttcttgtttgacaagttcattaaagctgctctagaagcgctaaaaactggagatattcttgccgttccagatggtgcgaaggccttcgaaaagctgggcttagatctcaaacttgtagacagtaagcctggtcaaccaatcaagaaggacactactagtgagcaacttaaagcacgatttacagttgaaggcgatgctactgctttcgattatttcgttgcatatgtgcgaatgtatctaaagggacaggacagtgaacccactccgtctctttaa

SEQ ID NO: 18 - protein, 213 aa, Treponema spp.
  SIGNAL (1)..(20)
MKKLRFLGFAIVAIALIVSCPGTSATPKASANTGGKVQPAPQPAPAPGESGNTSPDGNTPLGGTNDLSTEDVKKVVDKINQMFEGLGVEVTDSSPDAPITKETTPNQLKERFKFTKNPALADFLFDKFIKAALEALKTGDILAVPDGAKAFEKLGLDLKLVDSKPGQPIKKDTTSEQLKARFTVEGDATAFDYFVAYVRMYLKGQDSEPTPSL

SEQ ID NO: 19 - DNA, 1285 bp, Treponema spp.
  -35_signal (11)..(16)
  -10_signal (35)..(40)
aaaaacctacttgatatatatatatatatatatatataatagatagtaactttttattacaggaggatttcgtatgaaaaagttacgttttattagctttgccatattggcaacagcattgattgttgtatcttgtccaaggccaactgacaccccagaagtacctggcaatgttcagaaagctgaggagaaaaaagattctcccagtggaattcaaattaagatatccggtgatgaaagaatagataatacaagccttccagctcaaacggcaataaagggacaaaaatggaaagatgtaaaaacctctattgaagaaaaagtaaaattgagttacggttggaataatggcgactaccacatttaccaatggcgtctcggaaatgaaaatggcacgcttttaagcgacgactataagtttaccacgaatactactatttacgccgttaccaattatgcaaagtttaatcttgtggaaaatggtactgcaattgatgagtataaaggctatctcggtgaaaaacctcgtggcaaaataattatacccgaaggtgttacgaagattaatggtggagacaggagtggtgctttctctggttgtagcgggcttacaagcctcaccttgcctgacgggcttacatcgattggaattggtgctttcgttgattgtagcgggcttacgagcatcgacctttctgcatgcacaaagcttacctcgattgagtggggtgctttctatgattgtagcgggcttacaagcatcgacctttctgcatgcacaaagcttacctcgattggatttggtgctttcaggggttgtagcgggcttacaagcatcgacctttctgcatgcacaaagcttacctcgattggatttggtgctttcaggggttgtagcgggcttacaagcatcgacctttctgcatgcacaaagcttacctcgattgagtacgatactttctctggttgtagcgggcttacaagcatcgacctttctgcatgtacaaaacttacctcgattggaaacggtgctttctatgattgtagcgggcttacgagcatcaccttgcctgatgggcttacttcgattgggcactatgctttcgaaggttgtagcaagcttacttctgttacctttactaatagaaatggctgggcggtgtatgatgaccagaactataaaaacaagcatgcagatatatcgtcaagcggcttagatgatacagcaaaagccgctgatttgttaacaaccacatataaagataaatactggaaaaggaattag

SEQ ID NO: 20 - protein, 403 aa, Treponema spp.
  SIGNAL (1)..(21)
MKKLRFISFAILATALIVVSCPRPTDTPEVPGNVQKAEEKKDSPSGIQIKISGDERIDNTSLPAQTAIKGQKWKDVKTSIEEKVKLSYGWNNGDYHIYQWRLGNENGTLLSDDYKFTTNTTIYAVTNYAKFNLVENGTAIDEYKGYLGEKPRGKIIIPEGVTKINGGDRSGAFSGCSGLTSLTLPDGLTSIGIGAFVDCSGLTSIDLSACTKLTSIEWGAFYDCSGLTSIDLSACTKLTSIGFGAFRGCSGLTSIDLSACTKLTSIGFGAFRGCSGLTSIDLSACTKLTSIEYDTFSGCSGLTSIDLSACTKLTSIGNGAFYDCSGLTSITLPDGLTSIGHYAFEGCSKLTSVTFTNRNGWAVYDDQNYKNKHADISSSGLDDTAKAADLLTTTYKDKYWKRN